(19) United States
(12) Patent Application Publication
(10) Pub. No.: US 2022/0009961 A1
(43) Pub. Date: Jan. 13, 2022

(54) COMPOSITIONS, KITS AND METHODS USEFUL FOR SEPARATING PROTEINS FROM SURFACTANTS

(71) Applicant: Waters Technologies Corporation, Milford, MA (US)

(72) Inventor: Ximo Zhang, Framingham, MA (US)

(21) Appl. No.: 17/366,443

(22) Filed: Jul. 2, 2021

Related U.S. Application Data
(60) Provisional application No. 63/049,848, filed on Jul. 9, 2020.

(51) Int. Cl. C07K 1/34; C07K 14/005; B01D 21/26; B01D 21/00; B01D 15/08

(52) U.S. Cl. CPC C07K 1/34; C07K 14/005; C12N 2750/14051; B01D 21/0012; B01D 15/08; B01D 21/262

(57) ABSTRACT

The present disclosure pertains to methods for separating proteins from surfactants that comprise (a) adding an amount of denaturing buffer to a mixture comprising a protein and a surfactant thereby forming a denatured solution wherein the protein is denatured and (b) filtering the denatured solution with a molecular weight cutoff filter, thereby separating the protein from the surfactant and the denaturing buffer. The present disclosure also pertains to kits for forming such methods.

### CROSS-REFERENCE TO RELATED APPLICATIONS

This application claims the benefit of and priority to U.S. Provisional Patent Application No. 63/049,848, filed on Jul. 9, 2020, the entire contents of which is hereby incorporated by reference.

### FIELD

The present disclosure relates to compositions, kits and methods that may be used for removal of surfactants, including nonionic surfactants, from one or more proteins of interest, including capsid proteins.

### BACKGROUND

In recent years, gene therapy has become an emerging technology as a potential cure to many diseases, with adenoassociated virus (AAV) being the most commonly used carrier to deliver the therapeutic genes for treatment. The capsid of AAV particles contains three types of proteins, which should be well characterized and monitored to ensure drug safety and consistency.

A non-ionic surfactant, such as poloxamer or polysorbate surfactant, is commonly used in AAV-related bulk material and drug products to act as a stabilizer to prevent the AAV particles from aggregating or adsorbing onto container surfaces.

To characterize the AAV capsid proteins, however, it is preferable to remove these non-ionic surfactants prior to liquid chromatography-mass spectrometry (LC-MS) analysis, as they often interfere with the chromatographic separation of the proteins and can affect the MS ionization efficiency. Although the molecular weight of the surfactant is much smaller compared to the AAV capsid proteins, the hydrodynamic radius is comparable. Therefore it is difficult to remove the surfactant through traditional size-based methods, including dialysis, size exclusion chromatography, and molecular weight cut off filtration. In addition, the concentration and quantity of the AAV particles are at very low levels, resulting in low recovery when removing the surfactant from the intact AAV capsids.

### SUMMARY

In various aspects, the present disclosure is directed to the separation of proteins, including AAV capsid proteins among others, from surfactants, including polyoxyethylene-polyoxypropylene-polyoxyethylene triblock copolymer surfactants and polyoxyethylene sorbitan fatty acid ester surfactants, among others, under denatured conditions using a molecular weight cutoff filter. Without wishing to be bound by theory, under denaturing conditions, proteins typically unfold, resulting in larger hydrodynamic radii, whereas the hydrodynamic radii of surfactants remain approximately the same. This effect allows the protein to be separated from the surfactant in many cases.

In various aspects, the present disclosure is directed to methods for separating proteins from surfactants that comprise (a) adding an amount of denaturing buffer to a mixture comprising a protein and a surfactant thereby forming a denatured solution wherein the at least one protein is denatured and (b) filtering the denatured solution with a molecular weight cutoff filter, thereby separating the protein from the surfactant and the denaturing buffer.

In various embodiments, which may be used in conjunction with the above aspects, after separation from the surfactant, the protein is subjected to a chromatographic separation. In some embodiments the chromatographic separation is conducting using a combination of aqueous and organic mobile phases. In some of these embodiments, after chromatographic separation, the separated protein may be subjected to additional analytical techniques such as mass spectrometric analysis, fluorescence spectroscopy, ultraviolet spectroscopy, or combinations thereof, among others.

In various embodiments, which may be used in conjunction with the above aspects and embodiments, the surfactant is a non-ionic surfactant.

In various embodiments, which may be used in conjunction with the above aspects and embodiments, the protein may have a molecular weight ranging from 25 kDa or less to 200 kDa or more.

In various embodiments, which may be used in conjunction with the above aspects and embodiments, wherein the molecular weight cutoff filter has a molecular weight cutoff ranging from 10 kDa or less to 50 kDa or more.

In various embodiments, which may be used in conjunction with the above aspects and embodiments, the denaturing buffer comprises one or more organic solvents, water, and one or more acids and/or the denaturing buffer has a pH ranging from about 0 to 5.

In other aspects, the present disclosure pertains to kits for the separation of proteins from surfactants, which kits comprise a molecular weight cutoff filter, a protein denaturing buffer, and optionally one or more of the following: (a) a diluent buffer and/or (b) a dilution solution.

In various embodiments, which may be used in conjunction with the above aspects, the molecular weight cutoff filter of the kit may have a molecular weight cutoff ranging from 10 kDa or less to 50 kDa or more.

The above and other aspects and embodiments will further apparent to those of ordinary skill in the art upon review of the detailed description to follow.

### BRIEF DESCRIPTION OF THE DRAWINGS

FIG. 1A. is a deconvoluted MS spectra of eluted AAV proteins.

FIG. 1B is a deconvoluted MS spectra of the AAV proteins eluted at 2.79 min in FIG. 1A.

FIG. 1C is a chromatogram of eluted AAV proteins.

FIG. 2A a chromatogram of an unpurified dissociated AAV-8 capsid sample using reversed phase chromatography.

FIG. 2B is an expanded view of the chromatogram in the rectangle from FIG. 2A.

FIG. 3A is a chromatogram of purified dissociated AAV capsid sample using reversed phase chromatography.

FIG. 3B is an expanded view of the chromatogram in chromatogram of the rectangle from FIG. 3A.

FIG. 4A is an ESI MS spectra of a capsid protein, VP3, from an unpurified AAV-8 sample that containing surfactant poloxamer.

FIG. 4B is an ESI MS spectra of a capsid protein, VP2, from the unpurified sample that containing surfactant tween-20.

FIG. 5A is an ESI MS spectra of a capsid protein, VP3, from a purified AAV-8 sample.

FIG. 5B is an ESI MS spectra of capsid protein, VP2, from a purified AAV-5 sample.

### DETAILED DESCRIPTION

As previously noted, in various aspects, the present disclosure is directed to methods for separating at least one protein from at least one surfactant that comprises (a) adding an amount of denaturing buffer to a mixture comprising the at least one protein and the at least one surfactant, thereby forming a denatured solution wherein the at least one protein is denatured and (b) filtering the denatured solution with a molecular weight cutoff filter, thereby separating the at least one protein from the at least one surfactant and the denaturing buffer.

The surfactant may be non-ionic surfactant, for example, a non-ionic surfactant selected from polyoxyethylene sorbitan fatty acid esters, including polyoxyethylene sorbitan monolaurate (e.g., polysorbate 20), polyoxyethylene sorbitan monopalmitate (e.g., polysorbate 40), polyoxyethylene sorbitan monostearate (e.g., polysorbate 60), and polyoxyethylene sorbitan monooleate (e.g., polysorbate 80), polyoxyethylene-polyoxypropylene block copolymers, including polyoxyethylene-polyoxypropylene-polyoxyethylene triblock copolymers such poloxamer; polyoxyethylene alkylethers; polyethylene glycol fatty acids esters; polyethylene glycol glycerol fatty acid esters; polyglycerol fatty acid esters; polyoxyethylene glycerides; polyoxyethylene vegetable oils, or sodium dodecyl sulfate.

In various embodiments, the surfactant in the mixture is in the form of micelles. In some of these embodiments, the micelles have a diameter ranging from 5 to 12 nm.

In various embodiments, the protein may have a molecular weight ranging from 25 kDa or less to 200 kDa or more, for example, ranging anywhere from 25 kDa to 50 kDa to 75 kDa to 100 kDa to 125 kDa to 150 kDa to 175 kDa to 200 kDa (in other words, ranging between any two of these values).

In various embodiments, the protein comprises one or more capsid proteins, for example, selected from one or more AAV capsid proteins or other viral proteins such as adenovirus capsid proteins, one or more plasma proteins including serum albumin proteins, or other proteins with similar molecular weights.

In various embodiments, the molecular weight cutoff filter has a molecular weight cutoff ranging from 10 kDa or less to 50 kDa or more, for example, ranging anywhere from 10 kDa to 15 kDa to 20 kDa to 25 kDa to 30 kDa to 35 kDa to 40 kDa 45 kDa to 50 kDa.

In various embodiments, the molecular weight cutoff filter is selected such that it has a molecular weight cutoff ranging from about 5% to 80%, for example, ranging from 5% to 10% to 20% to 30% to 40% to 50% to 60% to 70% to 80% (i.e., ranging between any two of the preceding values) of a molecular weight of the protein(s) to be separated.

In various embodiments, the denaturing buffer may have a pH ranging from about 0 to 7, more typically 0 to 5, and in certain embodiments, having a pH ranging from about 2.0 to about 2.5.

In various embodiments, the denaturing buffer may comprise one or more organic solvents, water, and one or more acids.

Examples of organic solvents may be selected, for instance, from one or more of the following organic solvents: acetonitrile, isopropyl alcohol, ethanol, methanol, acetone, dichloromethane, tetrahydrofuran, methylene chloride, methyl ethyl ketone, DMSO or butyl alcohol. In certain embodiments, the one or more organic solvents comprise acetonitrile. In certain embodiments, the one or more organic solvents comprise acetonitrile (e.g., in an amount ranging from 5 to 80% (v/v)) and isopropanol (e.g., in an amount ranging from 5 to 80% (v/v)).

Examples of acids may be selected, for instance, from one or more of the following acids: halogenated (e.g., chlorinated or fluorinated) alkyl organic acids such as trichloroacetic acid, trifluoroacetic acid, pentafluoroproprionic acid, and heptafluorobutyric acid, non-halogenated alkyl organic acids such as formic acid, acetic acid, or propanoic acid, and inorganic acids such as sulfuric acid, hydrochloric acid, nitric acid, or phosphoric acid. In certain embodiments, the one or more acids comprise trifluoroacetic acid and formic acid. In certain embodiments, the one or more acids comprise trifluoroacetic acid, formic acid, and acetic acid. In certain embodiments, the one or more acids comprise 0.04 to 0.25% (v/v) trifluoroacetic acid, 0.04 to 0.25% (v/v) formic acid, and 4 to 25% (v/v) acetic acid.

In various embodiments, the molecular weight cutoff filter may be selected from a centrifugal filter, a positive pressure-driven filter or a vacuum-driven filter.

In various embodiments, the methods of the present disclosure further comprise adding an additional amount of denaturing buffer to the separated protein thereby forming a further denatured solution, after which the further denatured solution is filtered with the molecular weight cutoff filter, thereby separating the protein from the surfactant and the further denaturing buffer.

In various embodiments, the method further comprises adding a diluent buffer to the separated protein thereby forming a diluted solution, and then filtering the diluted solution with the molecular weight cutoff filter, thereby separating the protein from the diluent buffer. In certain embodiments, the diluent buffer comprises an organic acid and a suitable buffer such as a tris(hydroxymethyl) aminomethane buffer (Tris buffer) or HEPES (4-(2-hydroxyethyl)-1-piperazineethanesulfonic acid) buffer. In certain embodiments, the diluent buffer has a pH ranging from 0 to 7. In certain embodiments, the diluent buffer comprises acetic acid (e.g., 4-25% (v/v)) in Tris buffer (e.g., 8 to 50 mM).

In various embodiments, the separated protein is subsequently subjected to a chromatographic separation. The separation can be conducted by using isocratic or gradient elution with aqueous and organic solvents as the mobile phases, such as water and acetonitrile with 0.1% formic acid or the mobile phase additives.

In some of these embodiments, after chromatographic separation, the separated protein may be subjected to additional analytical techniques such as mass spectrometric analysis, fluorescence spectroscopy, ultraviolet spectroscopy, or combinations thereof, among others.

In other aspects, the present disclosure pertains to kits for the separation of proteins from surfactants, which kits comprise a molecular weight cutoff filter, a protein denaturing buffer, and optionally one or more of the following: (a) a diluent buffer or a dilution solution.

In various embodiments, the denaturing buffer may have a pH ranging from about 0 to 7, in some embodiments having a pH ranging from about 2.0 to about 2.5.

In various embodiments, the denaturing buffer may comprise one or more organic solvents, water, and one or more acids, which may be selected from those set forth above.

In various embodiments, diluent buffer my further comprise an organic acid and a suitable buffer, which may be selected from those set forth above.

### EXAMPLE

In the example to follow, AAV capsid proteins are removed from polyoxyethylene-polyoxypropylene-polyoxyethylene triblock copolymer surfactant (poloxamer) or polyoxyethylene sorbitan fatty acid ester surfactant (polysorbate 20). It should be noted, however, that the compositions, kits and methods described herein are applicable to a broad range of proteins other than AAV capsid proteins and a broad range of surfactants other than poloxamer and polysorbate surfactants.

Denaturing buffer is prepared in the following fashion: 20% (v/v) isopropanol, 10% (v/v) acetonitrile, 0.1% (v/v) trifluoroacetic acid, and 0.1% (v/v) formic acid in water; then add glacial acetic acid or an acetic acid solution to a final concentration of 10% (v/v).

Sample diluent buffer is prepared in the following fashion: 10% (v/v) acetic acid in 20 mM Tris buffer.

Using the preceding solutions, the separation is conducted as follows:
Step 1: add 50 μL dissociated AAV capsid sample to a molecular weight cutoff spin filter (Amicon 0.5 mL, 30-kDa cutoff, from MiliporeSigma, Burlington, Mass., USA). The AAV samples were purchased or obtained from collaborators and dissociated by the following procedures: add acetic acid in the received AAV sample to a final concentration of 10% and incubating at room temperature for 15 min.
Step 2: add 150 μL of denaturing buffer in the spin filter, spin down using a centrifuge at 14,000 rpm for 5 minutes.
Step 3: step 2 may be repeated once or twice, depending on the concentration of the surfactant.
Step 4: add 150 μL of sample diluent in the spin filter, spin down using a centrifuge at 14,000 rpm for 5 minutes.
Step 5: step 4 may be repeated.
Step 6: in a new collection tube, place the spin filter upside down, and collect the sample by centrifuging at 1,000 rpm for 2 minutes.

The unpurified and purified protein were analyzed using reversed phase chromatography, liquid chromatography/Ultraviolet (LC/UV) analysis, and liquid chromatography/mass spectroscopy (LC/MS) analysis.

A volume of 10 μL AAV capsid protein sample (approximately 0.5 μg proteins) was injected for each analysis. The experimental parameters are listed as below:

| | |
|---|---|
| Analytical | Waters BioAccord ™ LC-MS System incorporating: |
| system: | Waters ACQUITY UPLC ™ I-Class PLUS |
| | Waters ACQUITY ™ TUV Detector (280 nm) |
| | Waters ACQUITY RDa ™ MS Detector |
| LC column: | Waters ACQUITY ™ BEH C4 column, 1.7 μm,, |
| | 300 Å 2.1 × 100 mm |
| Column temp: | 80° C. |
| Mobile phase A: | LC-MS grade water with 0.1% DFA |
| Mobile phase B: | LC-MS grade acetonitrile with 0.1% DFA |

### Gradient Table

| Time | Flow rate | | |
|---|---|---|---|
| (min) | (mL/min) | % A | % B |
| Initial | 0.200 | 80.0 | 20.0 |
| 1.00 | 0.200 | 68.0 | 32.0 |
| 16.00 | 0.200 | 64.0 | 36.0 |
| 20.00 | 0.200 | 20.0 | 80.0 |
| 21.50 | 0.200 | 20.0 | 80.0 |
| 22.00 | 0.200 | 80.0 | 20.0 |
| 30.00 | 0.200 | 80.0 | 20.0 |

### ACQUITY RDa Detector Settings

| Mass range: | 400-7,000 | m/z |
|---|---|---|
| Mode: | ESI Positive | |
| Sampling rate: | 2 | Hz |
| Cone voltage: | 65 V for full scan | |
| Desolvation temp: | 550° | C. |
| Capillary voltage: | 1.5 | kV |

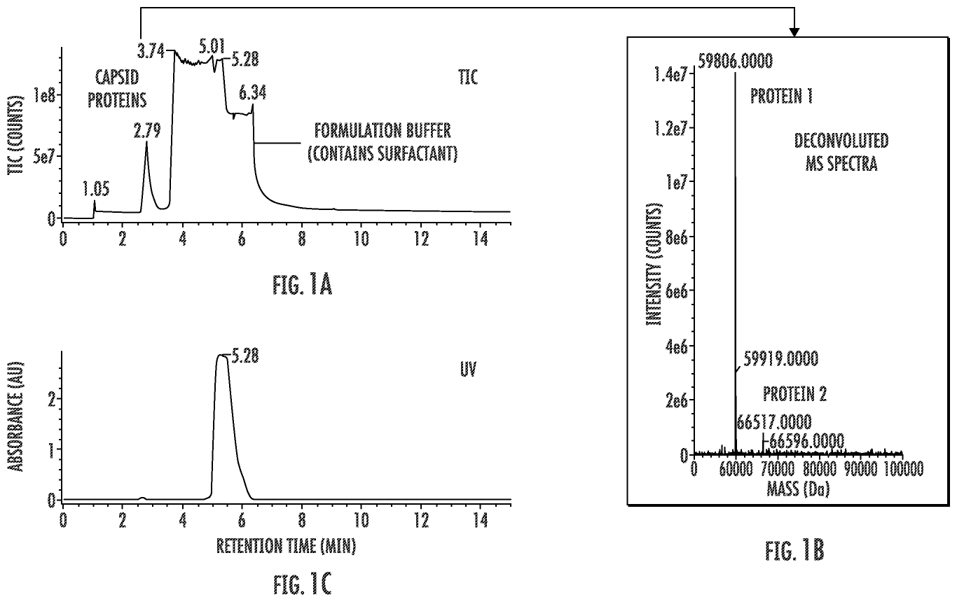

Using denatured size exclusion chromatography, the capsid proteins (e.g., AAV-5) were separated from the surfactant-containing buffer. The mobile phase was the same as the denaturing buffer described in “A Platform Method for the Molecular Mass Analysis of the Light Chains and Heavy Chains of Monoclonal Antibodies using the BioAccord System” Henry Shion, Ying Qing Yu, and Weibin Chen, Waters Technology Note, Waters Corporation, Milford, Mass., USA. FIG. 1A shows an expanded MS spectra, whereas FIG. 1B shows the deconvoluted MS spectra of the proteins eluted at 2.79 min in FIG. 1A, with FIG. 1B showing the masses of the AAV capsid proteins such as VP1 (labeled as protein 1) and VP2 (labeled as protein 2).

FIG. 1C is a UV chromatogram of the separation in FIG. 1A, showing the elution of the AAV capsid proteins and buffer.

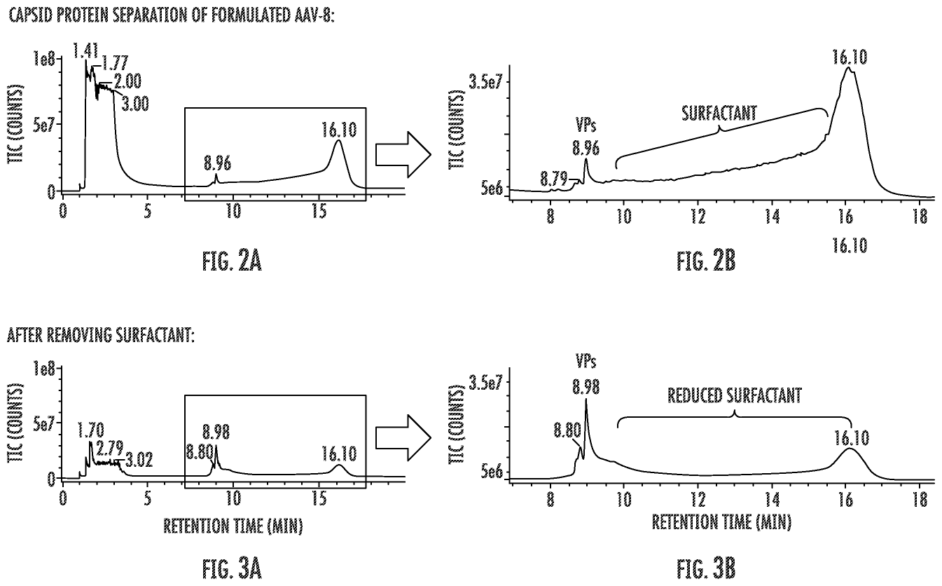

FIG. 2A is a chromatogram of the separation of the unpurified dissociated AAV-8 capsid sample using reversed phase chromatography. The peaks eluted before 5 min were from the buffer in the sample. The region marked by the rectangle showed the separation of the AAV capsid proteins and the surfactant.

FIG. 2B is an expanded view of the chromatogram in the rectangle from FIG. 2A. The rising baseline showed the impact of the surfactant on the separation of the AAV capsid proteins.

FIG. 3A is a chromatogram of the separation of the purified dissociated AAV-8 capsid sample using reversed phase chromatography. The buffer peaks eluted before 5 min were reduced, while the region marked by the rectangle showed the improved separation of the AAV capsid proteins.

FIG. 3B is an expanded view of the chromatogram in rectangle from FIG. 3A. The reduced baseline demonstrated the removal of the surfactant in the sample.

FIG. 4A is an ESI MS spectra of one of the capsid proteins, VP3, from the unpurified AAV-8 sample that containing surfactant poloxamer.

FIG. 4B is an ESI MS spectra of one of the capsid proteins, VP2, from the unpurified sample that containing surfactant tween-20.

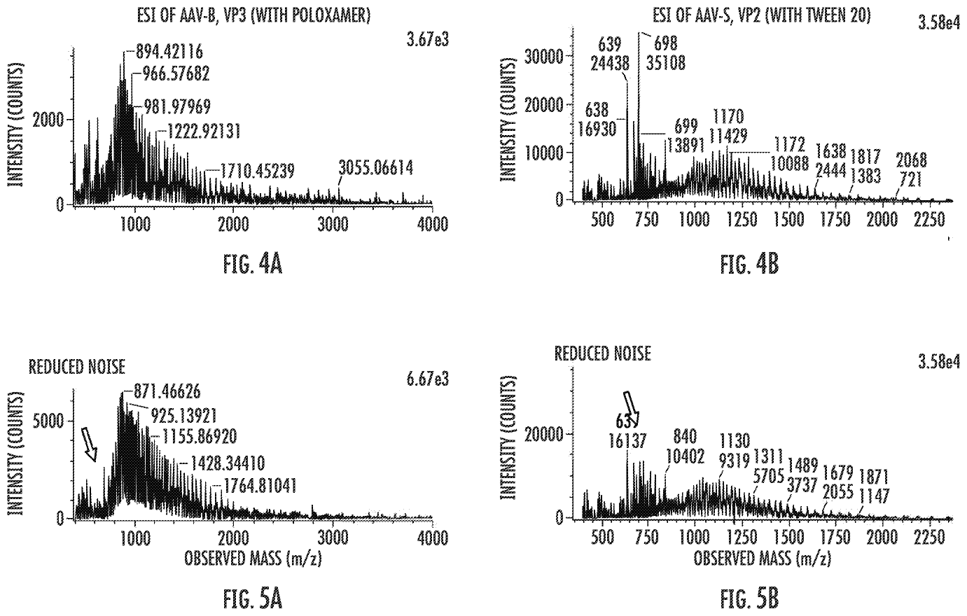

FIG. 5A is an ESI MS spectra of one of the capsid proteins, VP3, from the purified AAV-8 sample, showing the reduced noise under 1,000 m/z.

FIG. 5B is an ESI MS spectra of one of the capsid proteins, VP2, from the purified AAV-5 sample, showing the reduced noise under 750 m/z.

1. A method for separating proteins from surfactants that comprise (a) adding an amount of denaturing buffer to a mixture comprising a protein and a surfactant thereby forming a denatured solution wherein the protein is denatured and (b) filtering the denatured solution with a molecular weight cutoff filter, thereby separating the protein from the surfactant and the denaturing buffer.

2. The method of claim 1, wherein the surfactant is a non-ionic surfactant.

3. The method of claim 2, wherein the non-ionic surfactant is selected from polyoxyethylene sorbitan fatty acid esters and polyoxyethylene-polyoxypropylene block copolymers.

4. The method of claim 1, wherein the surfactant in the mixture is in the form of micelles.

5. The method of claim 1, wherein the protein has a molecular weight ranging from 25 kDa to 200 kDa.

6. The method of claim 1, wherein the protein comprises one or more of the following: capsid proteins or plasma proteins.

7. The method of claim 1, wherein the molecular weight cutoff filter has a molecular weight cutoff ranging from 10 kDa to 50 kDa, wherein the molecular weight cutoff filter has a molecular weight cutoff ranging from 5% to 80% of a molecular weight of the protein, or both.

8. The method of claim 1, wherein the denaturing buffer has a pH ranging from about 0 to 7.

9. The method of claim 1, wherein the denaturing buffer comprises one or more organic solvents, water, and one or more acids.

10. The method of claim 9, wherein the one or more organic solvents comprise acetonitrile

11. The method of claim 9, wherein the one or more acids comprise a halogenated organic acid and an alkyl organic acid.

12. The method of claim 9, wherein the one or more organic solvents comprise acetonitrile and isopropanol, and wherein the one or more acids comprise trifluoroacetic acid, formic acid and acetic acid.

13. The method of claim 1, the method further comprises adding an additional amount of denaturing buffer to the separated protein thereby forming a further denatured solution and filtering the further denatured solution with the molecular weight cutoff filter, thereby separating the protein from the surfactant and the additional amount of denaturing buffer.

14. The method of claim 1, wherein the method further comprises adding a diluent buffer to the separated protein thereby forming a diluted solution, and then filtering the diluted solution with the molecular weight cutoff filter, thereby separating the protein from the diluent buffer.

15. The method of claim 1, wherein separated protein is subsequently subjected to a liquid chromatographic separation step.

16. A kit for the separation of at least one protein from at least one surfactant, which comprise: a molecular weight cutoff filter and a protein denaturing buffer.

17. The kit of claim 16, wherein the molecular weight cutoff filter has a molecular weight cutoff ranging from 10 kDa to 50 kDa, wherein the molecular weight cutoff filter has a molecular weight cutoff ranging from 5 to 80% of a molecular weight of the protein or both.

18. The kit of any of claim 16, wherein the molecular weight cutoff filter is a centrifugal filter.

19. The kit of any of claim 16, wherein the denaturing buffer has a pH ranging from about 0 to 7.

20. The kit of any of claim 16, wherein the denaturing buffer comprises one or more organic solvents, water, and one or more acids.
